(19) United States
(12) Patent Application Publication
(10) Pub. No.: US 2025/0054143 A1
(43) Pub. Date: Feb. 13, 2025

(54) METHOD AND APPARATUS FOR VISUALIZING STRUCTURAL INFORMATION OF PULMONARY VESSELS IN MEDICAL IMAGES

(71) Applicant: CORELINE SOFT CO., LTD., Seoul (KR)

(72) Inventors: Donghoon YU, Gimpo-si (KR); Seungbin BAE, Goyang-si (KR); Sola SEO, Seoul (KR)

(21) Appl. No.: 18/800,105

(22) Filed: Aug. 11, 2024

(30) Foreign Application Priority Data
Aug. 11, 2023 (KR) 10-2023-0105223

(51) Int. Cl. G06T 7/00; G06T 7/10; G16H 50/20

(52) U.S. Cl. CPC G06T 7/0012; G06T 7/10; G16H 50/20; G06T 2207/30061; G06T 2207/30101

(57) ABSTRACT

A method of visualizing structural information of pulmonary vessels in medical images includes obtaining structural information of pulmonary vessels based on anatomical structure features of the pulmonary vessels from a medical image based on segmentation results of a pulmonary vascular region of the medical image; obtaining pulmonary embolism finding information in the pulmonary vessels of the medical image; visualizing the structural information of the pulmonary vessels in accordance with hierarchical information; and visualizing the pulmonary embolism finding information in association with individual sub-regions of the structural information of the pulmonary vessels visualized in accordance with the hierarchical information.

### CROSS-REFERENCE TO RELATED APPLICATION

This application claims priority from Korean Patent Application No. 10-2023-0105223 filed on Aug. 11, 2023, which is incorporated herein by reference in its entirety.

### TECHNICAL FIELD

The present invention relates to technology for processing, analyzing, and visualizing medical images, and more particularly to technology for generating and visualizing assistance information for the diagnosis of a pulmonary embolism.

### RELATED ART

The contents described in this section merely provide information about the background art of the present invention and do not constitute prior art.

Currently, medical images such as computed tomography (CT) images are widely used for diagnosis by analyzing lesions. For example, chest CT images are frequently used for diagnosis because they can be used to observe abnormalities in the inside of the body, such as the lungs, the bronchial tubes, the heart, etc.

A pulmonary embolism (PE) is a phenomenon in which a pulmonary artery is obstructed due to a solid substance (an embolus) that is usually formed by a blood clot or, rarely, by the accumulation of another substance.

U.S. Pat. No. 7,447,344 B2 entitled “System and Method for Visualization of Pulmonary Emboli from High-Resolution Computed Tomography Images” discloses a configuration for visualizing the tree structure of pulmonary vessels to visualize a pulmonary embolism.

U.S. Patent Application Publication No. US 2023-0081305 A1 entitled “Systems, Methods, and Apparatuses for Systematically Determining an Optimal Approach for the Computer-Aided Diagnosis of a Pulmonary Embolism” discloses a technology for detecting and classifying a pulmonary embolism using an image-level classification algorithm by using an artificial neural network.

The conventional technologies focus on detecting the presence/absence of a pulmonary embolism without error in a CT image or the like. Meanwhile, the vascular structure of pulmonary arteries is a structure that spreads out like tree branches. Therefore, even when visualized as a 3D model, blood vessels appear clumped together, so that a problem arises in that it is difficult to determine at a glance the part of a vascular structure in which a pulmonary embolism is located.

### SUMMARY

In order to diagnose a pulmonary embolism or determine the treatment effect of an antithrombotic agent or thrombolysis for a patient having a pulmonary embolism, information such as a quantitative embolism volume according to the location of a blood vessel is required.

The conventional technologies focus on detecting the presence/absence of a pulmonary embolism without error in a CT image or the like. Meanwhile, the vascular structure of pulmonary arteries is a structure that spreads out like tree branches. Accordingly, even when visualized as a 3D model, blood vessels appear to be clumped together, so that a problem arises in that it is difficult to determine at a glance the part of a vascular structure in which a pulmonary embolism is located.

An object of the present invention is to provide a user interface that may categorize/classify and visualize finding information for the diagnosis of a pulmonary embolism at the level of the segmental arteries of pulmonary vessels and effectively shows the possibility that there is a pulmonary embolism at a specific location of an overall pulmonary vascular structure.

An object of the present invention is to provide an effective diagnostic assistance user interface that may intuitively convey the location of pulmonary embolism finding information to a medical expert by visualizing the structural information of pulmonary vessels at the level of anatomical knowledge.

An object of the present invention is to effectively assist the diagnosis of a pulmonary embolism by providing various types of pulmonary embolism finding information including quantitative information together with the structural information of pulmonary vessels to a medical expert.

An object of the present invention is to provide the user interface for visualizing actual vascular structural information at the level of segmental arteries by using a simplified vascular structure diagram image generated based on the actual vascular structure of a patient.

An object of the present invention is to assist diagnosis by visualizing the pulmonary embolism finding information of each sub-region using a simplified vascular structure diagram image generated based on the actual vascular structure of a patient.

According to an aspect of the present invention, there may be provided a method of visualizing structural information of pulmonary vessels in medical images, the method comprising: obtaining structural information of pulmonary vessels based on anatomical structure features of the pulmonary vessels from a medical image based on segmentation results of a pulmonary vascular region of the medical image; obtaining pulmonary embolism finding information in the pulmonary vessels of the medical image; visualizing the structural information of the pulmonary vessels in accordance with hierarchical information; and visualizing the pulmonary embolism finding information in association with individual sub-regions of the structural information of the pulmonary vessels visualized in accordance with the hierarchical information.

In the method according to the present invention, the pulmonary embolism finding information visualized in each of the individual sub-regions of the structural information of the pulmonary vessels may include quantitative information about a possibility of a pulmonary embolism diagnosis for each sub-region of the structural information of the pulmonary vessels. The quantitative information may be a pulmonary embolism (PE) score, and may include quantitative information about the size of a pulmonary embolism and the degree of obstruction of the pulmonary embolism.

In the method according to the present invention, the pulmonary embolism finding information visualized in each of the individual sub-regions of the structural information of the pulmonary vessels may include whether an embolus has been detected in each sub-region of the structural information of the pulmonary vessels and a size of the detected embolus.

In the method according to the present invention, the obtaining structural information of the pulmonary vessels may comprise obtaining segmentation information for each of the individual sub-regions including a left main artery, a right main artery, lobar arteries and segmental arteries of the structural information of the pulmonary vessels.

In the method according to the present invention, the visualizing the structural information of the pulmonary vessels may comprise visualizing the individual sub-regions of the structural information of the pulmonary vessels in accordance with hierarchical information ranging up to a left main artery, a right main artery, lobar arteries, and segmental arteries.

In the method according to the present invention, the obtaining the structural information of the pulmonary vessels may comprise obtaining the structural information of the pulmonary vessels based on the anatomical structure features of the pulmonary vessels based on at least one of whether the pulmonary vessels have branched from a main artery trunk toward segmental arteries or results of comparing relative sizes of the sub-regions of the pulmonary vessels before the branching and relative sizes of the sub-regions of the pulmonary vessels after the branching.

In the method according to the present invention, the obtaining the structural information of the pulmonary vessels may comprise obtaining the structural information of the pulmonary vessels including a distribution of individual segmental arteries by using at least one of the segmentation results of the pulmonary vascular region, segmentation results of lobes of a lung region of the medical image, or previously known anatomical structure features of the pulmonary vessels.

In the method according to the present invention, the pulmonary embolism finding information visualized in each of the individual sub-regions of the structural information of the pulmonary vessels may include a Qanadli score that quantifies a number of emboli associated with each sub-region of the structural information of the pulmonary vessels.

In the method according to the present invention, the pulmonary embolism finding information visualized in each of the individual sub-regions of the structural information of the pulmonary vessels may include a Miller score using an objective score based on a segmental embolism for each sub-region of the structural information of the pulmonary vessels and a subjective score based on reduction of peripheral perfusion.

According to an aspect of the present invention, there may be provided an apparatus for visualizing structural information of pulmonary vessels in medical images, the apparatus comprising: memory configured to store one or more instructions; and a processor configured to load the one or more instructions from the memory and execute the one or more instructions.

In the apparatus according to the present invention, the processor, by executing the one or more instructions, may be configured to: obtain structural information of pulmonary vessels based on anatomical structure features of the pulmonary vessels from a medical image based on segmentation results of a pulmonary vascular region of the medical image; obtain pulmonary embolism finding information in the pulmonary vessels of the medical image; visualize the structural information of the pulmonary vessels in accordance with hierarchical information; and visualize the pulmonary embolism finding information in association with individual sub-regions of the structural information of the pulmonary vessels visualized in accordance with the hierarchical information.

In the apparatus according to the present invention, the pulmonary embolism finding information visualized in each of the individual sub-regions of the structural information of the pulmonary vessels may include quantitative information about a possibility of a pulmonary embolism diagnosis for each sub-region of the structural information of the pulmonary vessels.

In the apparatus according to the present invention, the pulmonary embolism finding information visualized in each of the individual sub-regions of the structural information of the pulmonary vessels may include whether an embolus has been detected in each sub-region of the structural information of the pulmonary vessels and a size of the detected embolus.

In the apparatus according to the present invention, the processor, by executing the one or more instructions, may be further configured to obtain segmentation information for each of the individual sub-regions including a left main artery, a right main artery, lobar arteries and segmental arteries of the structural information of the pulmonary vessels when obtaining the structural information of the pulmonary vessels.

In the apparatus according to the present invention, the processor, by executing the one or more instructions, may be further configured to visualize the individual sub-regions of the structural information of the pulmonary vessels in accordance with hierarchical information ranging up to a left main artery, a right main artery, lobar arteries, and segmental arteries when visualizing the structural information of the pulmonary vessels in accordance with the hierarchical information.

In the apparatus according to the present invention, the processor, by executing the one or more instructions, may be further configured to obtain the structural information of the pulmonary vessels based on the anatomical structure features of the pulmonary vessels based on at least one of whether the pulmonary vessels have branched from a main artery trunk toward segmental arteries or results of comparing relative sizes of the sub-regions of the pulmonary vessels before the branching and relative sizes of the sub-regions of the pulmonary vessels after the branching when obtaining the structural information of the pulmonary vessels.

In the apparatus according to the present invention, the processor, by executing the one or more instructions, may be further configured to obtain the structural information of the pulmonary vessels including a distribution of individual segmental arteries by using the segmentation results of the pulmonary vascular region, segmentation results of lobes of a lung region of the medical image, or previously known anatomical structure features of the pulmonary vessels when obtaining the structural information of the pulmonary vessels.

According to an embodiment of the present invention, finding information for the diagnosis of a pulmonary embolism may be categorized/classified and visualized at the level of the segmental arteries of pulmonary vessels.

According to an embodiment of the present invention, there may be provided the user interface that effectively shows the possibility that there is a pulmonary embolism at a specific location of an overall pulmonary vascular structure.

According to an embodiment of the present invention, there may be provided the effective diagnostic assistance user interface that may intuitively convey the location of pulmonary embolism finding information to a medical expert by visualizing the structural information of pulmonary vessels at the level of anatomical knowledge.

According to an embodiment of the present invention, it may be possible to effectively assist the diagnosis of a pulmonary embolism by providing various types of pulmonary embolism finding information including quantitative information together with the structural information of pulmonary vessels to a medical expert.

According to an embodiment of the present invention, there may be provided the user interface for visualizing actual vascular structural information at the level of segmental arteries by using a simplified vascular structure diagram image generated based on the actual vascular structure of a patient.

According to an embodiment of the present invention, diagnosis may be assisted by visualizing the pulmonary embolism finding information of each sub-region using a simplified vascular structure diagram image generated based on the actual vascular structure of a patient.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a conceptual view showing a generalized user interface for visualizing pulmonary arteries and pulmonary embolism finding information in a medical image;

FIG. 2 is an operational flowchart showing a method of visualizing the structural information of pulmonary vessels in medical images according to an embodiment of the present invention;

FIG. 3 is an operational flowchart showing an embodiment of a process (step S100) of the method of FIG. 2 in more detail;

FIG. 4 is a conceptual diagram showing the anatomical structure information of pulmonary lobes used in an embodiment of the present invention;

FIG. 5 is a conceptual diagram showing a structured diagram for visualizing the structural information of pulmonary vessels in medical images according to an embodiment of the present invention;

FIGS. 6 and 7 are conceptual diagrams illustrating user interfaces that show quantitative information used in embodiments of the present invention; and

FIG. 8 is a conceptual diagram showing an example of a generalized apparatus for performing medical image processing, analysis and visualization, and diagnosis assistance and visualizing the finding information in medical images according to the embodiments of FIGS. 1 to 7.

### DETAILED DESCRIPTION

Other objects and features of the present invention in addition to the above-described objects will be apparent from the following description of embodiments to be given with reference to the accompanying drawings.

The embodiments of the present invention will be described in detail below with reference to the accompanying drawings. In the following description, when it is determined that a detailed description of a known component or function may unnecessarily make the gist of the present invention obscure, it will be omitted.

Relational terms such as first, second, and the like may be used for describing various elements, but the elements should not be limited by the terms. These terms are only used to distinguish one element from another. For example, a first component may be named a second component without departing from the scope of the present disclosure, and the second component may also be similarly named the first component. The term “and/or” means any one or a combination of a plurality of related and described items.

When it is mentioned that a certain component is “coupled with” or “connected with” another component, it should be understood that the certain component is directly “coupled with” or “connected with” to the other component or a further component may be disposed therebetween. In contrast, when it is mentioned that a certain component is “directly coupled with” or “directly connected with” another component, it will be understood that a further component is not disposed therebetween.

The terms used in the present disclosure are only used to describe specific exemplary embodiments, and are not intended to limit the present disclosure. The singular expression includes the plural expression unless the context clearly dictates otherwise. In the present disclosure, terms such as ‘comprise’ or ‘have’ are intended to designate that a feature, number, step, operation, component, part, or combination thereof described in the specification exists, but it should be understood that the terms do not preclude existence or addition of one or more features, numbers, steps, operations, components, parts, or combinations thereof.

Unless otherwise defined, all terms (including technical and scientific terms) used herein have the same meaning as commonly understood by one of ordinary skill in the art to which this disclosure belongs. Terms that are generally used and have been in dictionaries should be construed as having meanings matched with contextual meanings in the art. In this description, unless defined clearly, terms are not necessarily construed as having formal meanings.

Meanwhile, even when the technology was known before the filing date of the present application, it may be included as part of the configuration of the present invention of the present application when necessary, and this will be described herein to the extent that it does not obscure the purpose of the present invention. However, in the following description of the configuration of the present invention of the present application, detailed descriptions of items that can be clearly understood by those skilled in the art as the technologies known before the filing date of the present application may obscure the purpose of the present invention, so that excessively detailed descriptions of known technologies will be omitted.

For example, technologies known before the application of the present invention may be used as technology for detecting, segmenting, and classifying specific organs and sub-regions of the human body by processing medical images, technology for generating quantified information by measuring segmented organs or finding regions, and the like, and at least some of these known technologies may be applied as elemental technologies required for practicing the present invention. For example, descriptions of elemental technologies required for practicing parts of the configuration of the present invention may be replaced by providing notification that the technologies are known to those skilled in the art through U.S. Pat. No. 7,447,344 B2 entitled “System and Method for Visualization of Pulmonary Emboli from High-Resolution Computed Tomography Images” and U.S. Patent Application Publication No. US 2023-0081305 A1 entitled “Systems, Methods, and Apparatuses for Systematically Determining an Optimal Approach for the Computer-Aided Diagnosis of a Pulmonary Embolism.”

In the prior art literature, lesion candidates are detected using an artificial neural network, and findings are generated by classifying the lesion candidates. Each of the findings includes diagnosis assistance information. The diagnosis assistance information may include quantitative measurements such as the probability that each finding is actually a lesion, the confidence and malignity of the finding, and the sizes and volumes of lesion candidates to which the finding corresponds.

In medical image diagnosis support using an artificial neural network, each finding needs to include probability and quantified confidence as diagnosis assistance information. Since all findings may not be provided to a user, findings are generally filtered by applying a specific threshold, and only findings that are filtered out are provided to the user. In a workflow in which a clinical finding is generated in such a manner that a user who is a radiologist reads a medical image and the results of diagnosis are generated in such a manner that a clinician analyzes the finding, an artificial neural network or automated program may at least partially assist the radiologist in a reading process and a finding generation process and/or the clinician in a diagnosis process.

However, the purpose of the present invention is not to claim rights to these known technologies, and the content of the known technologies may be included as part of the present invention within the range that does not depart from the spirit of the present invention.

Hereinafter, embodiments of the present invention will be described in more detail with reference to the accompanying drawings. In order to facilitate overall understanding in the description of the present invention, the same reference numerals will be used for the same components throughout the drawings, and redundant descriptions of the same components will be omitted.

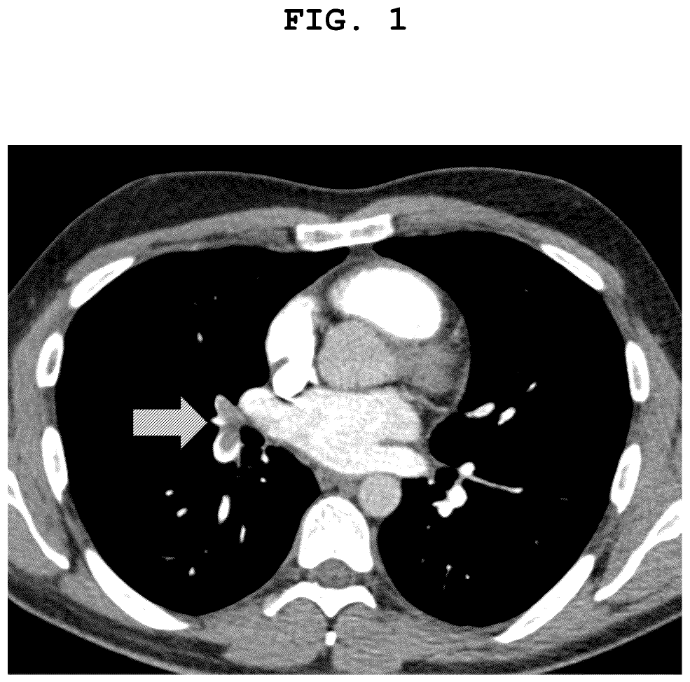

FIG. 1 is a conceptual view showing a generalized user interface for visualizing pulmonary arteries and pulmonary embolism finding information in a medical image.

FIG. 1 illustrates an example of a CT image using a contrast agent. Referring to FIG. 1, there is shown an embodiment in which a CT image is reconstructed into a 3D model and then the 3D model is shown on an axial plane. Since a pulmonary artery vessel structure is complex and appears for blood vessels to be clumped together, there is a problem in that the presence/absence of an embolus and the approximate size of the embolus may be determined using only the image shown in FIG. 1 but it is difficult to briefly determine the branch of the blood vessels in which the embolus is present.

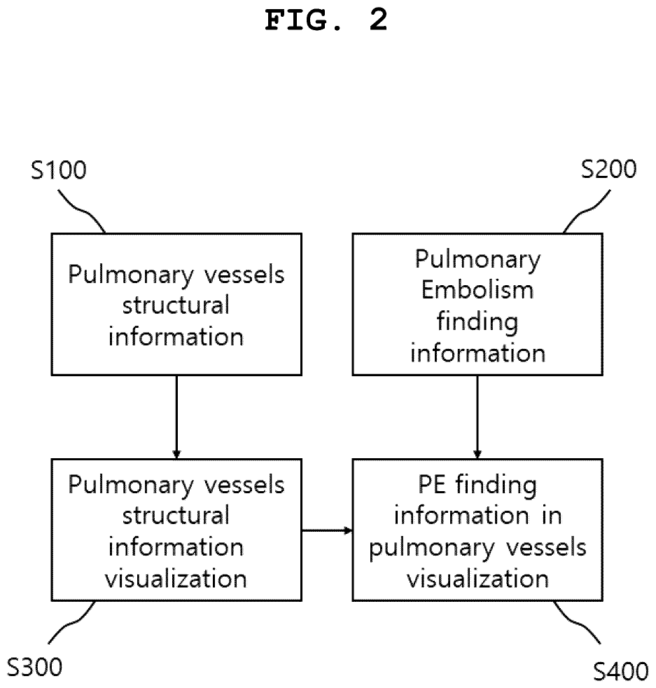

FIG. 2 is an operational flowchart showing a method of visualizing the structural information of pulmonary vessels in medical images according to an embodiment of the present invention.

Referring to FIG. 2, the method of visualizing the structural information of pulmonary vessels in medical images according to the present embodiment may include: step S100 of obtaining the structural information of pulmonary vessels based on anatomical structure features of the pulmonary vessels from a medical image based on segmentation results of a pulmonary vascular region of the medical image; step S200 of obtaining pulmonary embolism finding information in the pulmonary vessels of the medical image; step S300 of visualizing the structural information of the pulmonary vessels in accordance with hierarchical information; and step S400 of visualizing the pulmonary embolism finding information in association with the individual sub-regions of the structural information of the pulmonary vessels visualized in accordance with the hierarchical information.

The pulmonary embolism finding information visualized in each of the individual sub-regions of the structural information of the pulmonary vessels may include quantitative information about the possibility of a pulmonary embolism diagnosis for each sub-region of the structural information of the pulmonary vessels. The quantitative information may be a pulmonary embolism (PE) score, and may include quantitative information about the size of a pulmonary embolism and the degree of obstruction of the pulmonary embolism. In this case, the degree of obstruction may refer to the degree of obstruction in the sub-region in which corresponding pulmonary embolism finding information is located.

The pulmonary embolism finding information visualized in each of the individual sub-regions of the structural information of the pulmonary vessels may include whether an embolus has been detected in each sub-region of the structural information of the pulmonary vessels and the size of the detected embolus.

In step S100 of obtaining the structural information of pulmonary vessels, there may be obtained segmentation information for each of the individual sub-regions including a left main artery, a right main artery, lobar arteries and segmental arteries of the structural information of the pulmonary vessels.

As a blood vessel enters the left or right lung from the main artery trunk of the pulmonary vessels, it may be classified as the left or right main artery.

The branch formed when the left or right main artery branches off within the left or right lung may be classified as an interlobar artery, and may then be classified as a lobar artery, a segmental artery, or a sub-segmental artery while branching off within each lung lobe.

In step S300 of visualizing the structural information of the pulmonary vessels in accordance with hierarchical information, the individual sub-regions of the structural information of the pulmonary vessels may be visualized in accordance with hierarchical information ranging up to the left main artery, the right main artery, the lobar arteries, and the segmental arteries.

In an embodiment of the present invention, the pulmonary vessels may be divided/segmented and/or classified/categorized into sub-regions ranging from the interlobar arteries to the lobar arteries and/or the segmental arteries, and the pulmonary embolism finding information corresponding to each of the individual sub-regions may be visualized. In another embodiment of the present invention, sub-regions ranging up to not only the segmental arteries but also the sub-segmental arteries may be divided/segmented from each other and shown as needed.

In an embodiment of the present invention, an index image capable of showing sub-regions ranging at least from the interlobar arteries up to the lobar arteries and/or the segmental arteries on a single image may be provided via one of the user interfaces.

In step S100 of obtaining the structural information of pulmonary vessels, the structural information of the pulmonary vessels based on the anatomical structure features of the pulmonary vessels may be obtained based on at least one of 1) whether the pulmonary vessels have branched off from the main artery trunk toward the segmental arteries and/or 2) the results of comparing the relative sizes of the sub-regions of the pulmonary vessels before the branching and the relative sizes of the sub-regions of the pulmonary vessels after the branching.

In step S100 of obtaining the structural information of pulmonary vessels, the structural information of the pulmonary vessels including the distribution of individual segmental arteries may be obtained using at least one of 1) the segmentation results of the pulmonary vascular region, 2) the segmentation results of the lobes of the lung region of the medical image, and/or 3) the previously known anatomical structure features of the pulmonary vessels.

The pulmonary embolism finding information visualized in each of the individual sub-regions of the structural information of the pulmonary vessels may include a Qanadli score that quantifies the number of emboli associated with each sub-region of the structural information of the pulmonary vessels.

The pulmonary embolism finding information visualized in each of the individual sub-regions of the structural information of the pulmonary vessels may include a Miller score using an objective score based on a segmental embolism for each sub-region of the structural information of the pulmonary vessels and a subjective score based on reduction of peripheral perfusion.

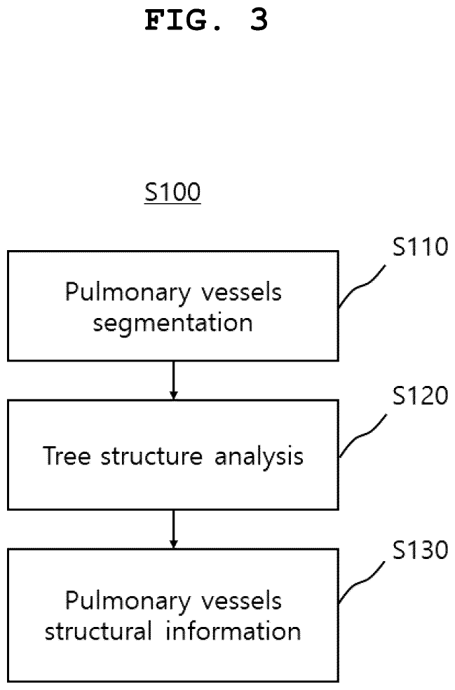

FIG. 3 is an operational flowchart showing an embodiment of a partial process (step S100) of the method of FIG. 2 in more detail.

Step S100 of obtaining the structural information of pulmonary vessels may include: step S110 of segmenting a pulmonary vascular region in the medical image; step S120 of analyzing the tree hierarchy of the individual sub-regions in the pulmonary vessels by taking into consideration the segmentation results of the pulmonary vessels and the previously known anatomical structure features of the pulmonary vessels; and step S130 of obtaining the structural information of the pulmonary vessels including the distribution of individual segmental arteries.

Step S110 of segmenting a pulmonary vascular region in the medical image may be performed independently from step S200 of obtaining pulmonary embolism finding information. In this case, steps S110 and S200 may be implemented in a rule-based manner, or may be implemented by processing/analyzing the medical image using an (pre-trained) artificial neural network.

Steps S120 and S130 may be implemented using the previously known knowledge of anatomical structures based on the results of step S110. This process may also be implemented in a rule-based manner, or may also be implemented by processing/analyzing the medical image using an (pre-trained) artificial neural network.

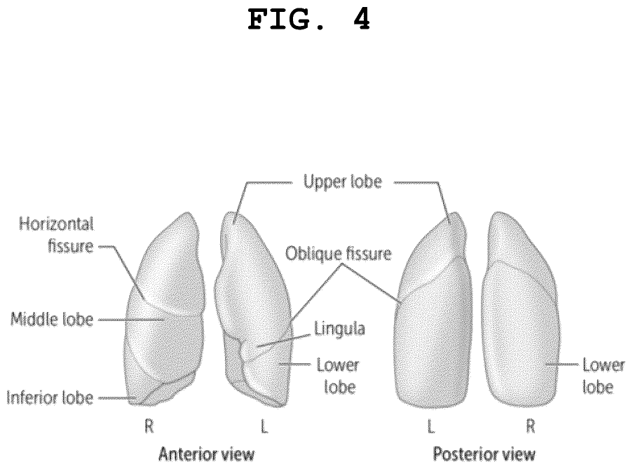

FIG. 4 is a conceptual diagram showing the anatomical structure information of pulmonary lobes used in an embodiment of the present invention.

Referring to FIG. 4, in general, the right lung may be divided/segmented/classified into the right upper lobe (RUL), the right middle lobe (RML), and the right lower lobe (RLL), and the left lung may be divided/segmented/classified into the left upper lobe (LUL), and the left middle lobe (LLL). Furthermore, the left lung may have the lingula region instead of the middle lobe, and the pulmonary vessels may be classified based on segmentation information for each of these pulmonary lobes.

An example of the previously known anatomical structure knowledge is that there are five segmental arteries located in the lower lobe, three segmental arteries in the upper lobe, and two segmental arteries in the middle lobe or lingula region.

When both the left lung and the right lung are taken into consideration, the number of segmental arteries may be considered to be approximately 20.

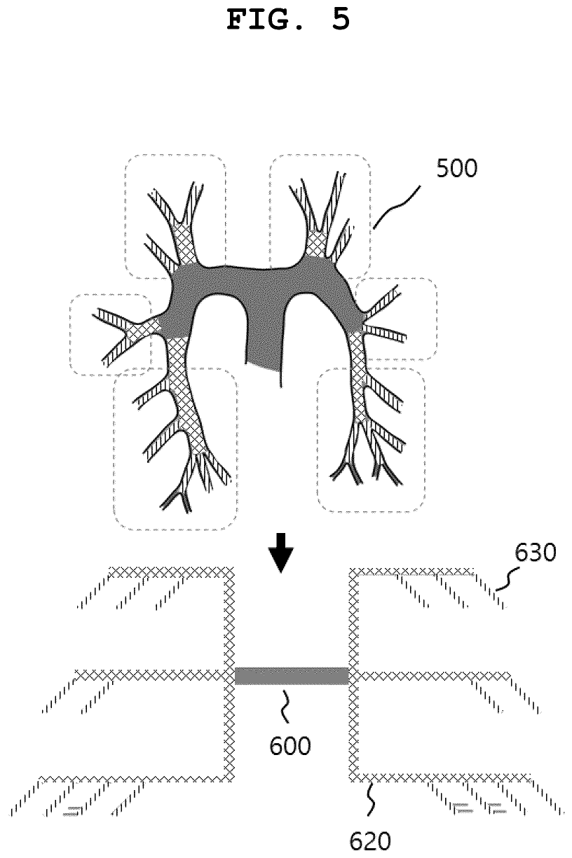

FIG. 5 is a conceptual diagram showing a structured diagram for visualizing the structural information of pulmonary vessels in medical images according to an embodiment of the present invention.

Referring to FIGS. 2 and 5 together, in step S100 of obtaining the structural information of pulmonary vessels, there may be obtained segmentation information for individual sub-regions including the left main artery, right main artery, lobar arteries and segmental arteries of the structural information of the pulmonary vessels.

In step S100 of obtaining the structural information of pulmonary vessels, the structural information of the pulmonary vessels including the distribution of individual segmental arteries may be obtained using at least one of 1) the segmentation results of the pulmonary vascular region (see the results of step S110), 2) the segmentation results of the lobes of the lung region of the medical image (see the conceptual diagram of FIG. 4), and/or 3) the previously known anatomical structure features of the pulmonary vessels (see the results of step S120) (refer to step S130).

As described above, as a blood vessel enters the left or right lung from the main artery trunk of the pulmonary vessels, it may be classified as the left or right main artery.

The branch formed when the left or right main artery branches off within the left or right lung may be classified as an interlobar artery, and may then be classified as a lobar artery, a segmental artery, or a sub-segmental artery while branching off within each lung lobe. In this case, generalized anatomical structure information for the left and right lungs, such as that shown in FIG. 4, may be used as information 500 about the lung lobes.

In step S300 of visualizing the structural information of the pulmonary vessels in accordance with hierarchical information, there may be shown a hierarchical structure in which the individual sub-regions of the structural information of the pulmonary vessels branch off from the main artery trunk 600. Although the main artery trunk 600 is classified as a single region in FIG. 5 for ease of description, the corresponding region may be divided/segmented and/or classified/categorized into and shown as the left main artery, the right main artery, and the interlobar arteries based on anatomical structure features and hierarchical structural information in another embodiment of the present invention.

Referring back to FIG. 5, as a result of step S300, the individual sub-regions of the structural information of the pulmonary vessels ranging from the main artery trunk 600 region to the lobar arteries 620 and the segmental arteries 630 may be visualized in a divided/segmented state in accordance with the hierarchical information.

In an embodiment of the present invention, the pulmonary vessels are divided/segmented into sub-regions ranging from the interlobar arteries to the lobar arteries 620 and the segmental arteries 630, and pulmonary embolism finding information corresponding to each of the sub-regions may be visualized. In another embodiment of the present invention, sub-regions ranging up to not only the segmental arteries 630 but also the sub-segmental arteries may be divided/segmented from each other and shown as needed. In general, the term “pulmonary vessels” refers to a concept including pulmonary arteries, the pulmonary veins, and bronchial arteries. In an embodiment of the present invention, the structural information of pulmonary vessels may be understood as information in which a tree hierarchy structure for the pulmonary arteries has been mainly identified. However, the spirit of the present invention is not intended to exclude the pulmonary veins and the bronchial arteries from the scope of the invention.

In an embodiment of the present invention, an index image capable of showing sub-regions ranging at least from the interlobar arteries up to the lobar arteries and the segmental arteries on a single image may be provided to one of the user interfaces. A simplified diagram image (an index image) such as that shown in the upper part of FIG. 5 may be provided as a conceptual diagram for the structural information of the pulmonary vessels, and the results of performing analysis by applying the conceptual diagram, shown in the upper part of FIG. 5, to an actual medical image may be provided as the results of step S400, such as the simplified diagram image (the index image) shown in the lower part of FIG. 5.

In step S100 of obtaining the structural information of pulmonary vessels, the structural information of the pulmonary vessels based on the anatomical structure features of the pulmonary vessels may be obtained based on at least one of 1) whether the pulmonary vessels branch from the main artery trunk toward the segmental arteries and/or 2) the results of comparing the relative sizes of the sub-regions of the pulmonary vessels before the branching and the relative sizes of the sub-regions of the pulmonary vessels after the branching.

That is, each of the sub-regions in the structural information of the pulmonary vessels may be determined to belong to one of the main artery trunk 600, the lobar arteries 620, and the segmental arteries 630 not only by whether it branches off from the main artery trunk 600 toward the segmental arteries 630 or the sub-segmental arteries, but also may be determined to belong to one of the main artery trunk 600, the lobar arteries 620, the segmental arteries 630, and the sub-segmental arteries by taking into consideration whether the branch to which the sub-region belongs is a main branch.

Accordingly, as shown in FIG. 5, even after the lobar arteries 620 have branched off from the main artery trunk 600, the main branch may still be determined to belong to the main artery trunk 600. In the same manner, even after the segmental arteries 630 have branched off from the lobar arteries 620, the main branch may still be classified as the lobar arteries 620.

The branch that branches off from one lobar artery 620 but is larger than the remaining branches that branched off is classified as a main branch, and may still be classified as a lobar artery 620. The remaining branches may be classified as segmental arteries 630, and the level thereof may be reduced by one or more levels in terms of a hierarchical structure.

According to an embodiment of the present invention, after at least one lobar artery 620 has branched off from the main artery trunk 600, the segmental arteries 630 may be determined to branch off directly from the main artery trunk 600. That is, the mapping of each of the sub-regions in the structural information of the pulmonary vessels in the hierarchical structure may be determined based on at least two criteria. In this case, when each sub-region is mapped onto the hierarchical structure, previously known expert knowledge about the anatomical structure of the pulmonary arteries may be additionally taken into consideration.

Referring to FIG. 5, hierarchical information in the anatomical structure of the pulmonary arteries may be shown using the line thickness, line type, shape size, and/or color/pattern of each segment in the diagram image (the index image).

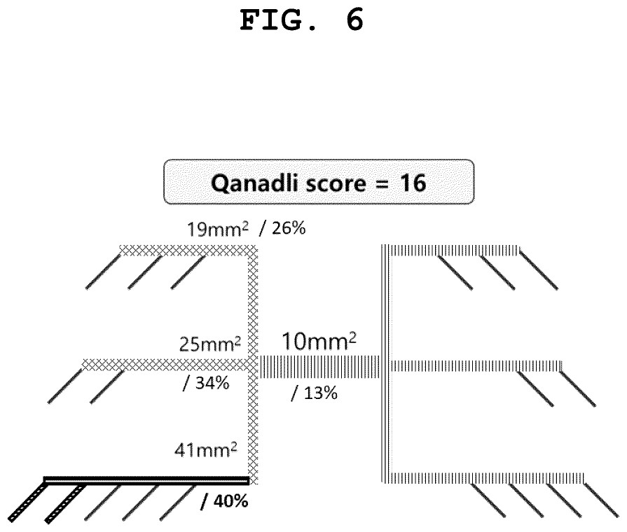

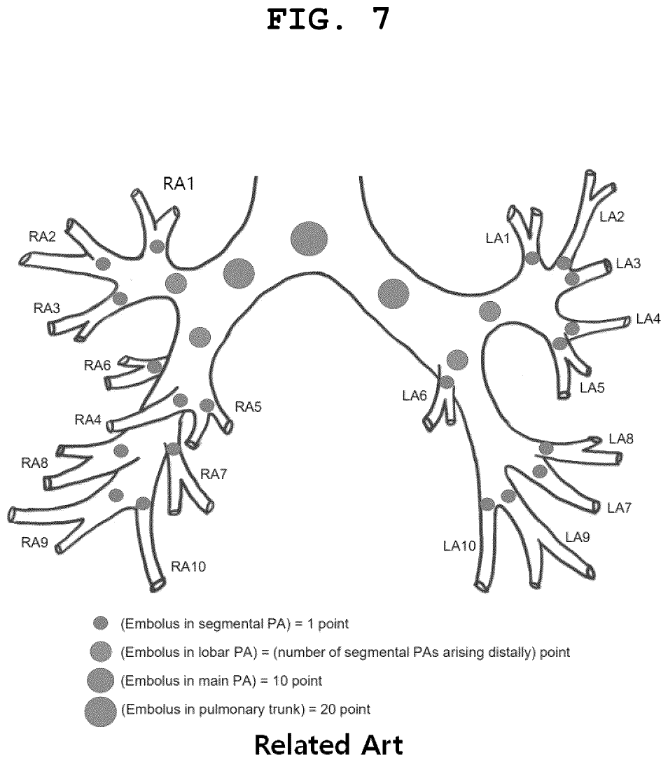

FIGS. 6 and 7 are conceptual diagrams illustrating user interfaces that show quantitative information used in embodiments of the present invention.

Referring to FIG. 6, there is shown a case in which a Qanadli score is mapped to and visualized for, as quantitative information, each sub-region in the structural information of pulmonary vessels.

Referring to FIG. 7, there is shown a process of calculating a Qanadli score on a conceptual diagram of the structural information of pulmonary vessels. The embodiments of the present invention may utilize the Schematic diagram of Qanadli scoring system shown in “Acute Pulmonary Embolism and Chronic Thromboembolic Pulmonary Hypertension: Clinical and Serial CT Pulmonary Angiographic Features”, Junho An et al, J Korean Med Sci., March 2022. As acknowledged, the disclosure of the prior art may be included as part of the configuration of the present invention to the extent that it does not obscure the purpose of the present invention.

Examples of the values that may be shown in the embodiment of FIG. 6 include the presence/absence of a pulmonary embolism, and the size of the pulmonary embolism, the degree of obstruction of each sub-region, and the calculated value of a pulmonary embolism (PE) score when located.

Referring to FIG. 6, hierarchical information in the anatomical structure of pulmonary arteries may be shown using the line thickness, line type, shape size, and/or color/pattern of each segment in the diagram image (the index image). The remaining visualization elements (the line thickness, line type, shape size, and/or color/pattern of each segment) that are not used to show the hierarchical information may be additionally visualized based on the magnitude of quantified information, clinical significance, and the result of comparison with a threshold.

The Qanadli score may be represented as follows when the number of segmental arteries affected by a pulmonary embolism is n.

$$Qanadli Score = \sum ( n \cdot d ) \qquad [ Equation 1 ]$$

In the above equation, d denotes the degree of vascular obstruction, and may be calculated as 0 points when there is no thrombus, 1 point when there is partial obstruction due to embolism (a partially occlusive thrombus), and 2 points when there is complete occlusion (total occlusion). Since the number of segmental arteries is assumed to be a maximum of 20, the maximum value of the Qanadli score is 40 points. As the score increases, the risk of pulmonary embolism may be diagnosed as being higher.

Referring to FIG. 7, there is shown a process in which the score d is calculated in each conceptually distinct branch of a pulmonary vessel, the score d is transferred to a higher hierarchical structure, and then a final score is calculated.

RA1 to RA3 denote segmental arteries belonging to the right upper lobe, RA4 to RA5 denote segmental arteries belonging to the right middle lobe, and RA6 to RA10 denote segmental arteries belonging to the right lower lobe.

LA1 to LA3 denote segmental arteries belonging to the left upper lobe, LA4 to LA5 denote segmental arteries belonging to the lingula region, and LA6 to LA10 denote segmental arteries belonging to the left lower lobe.

The embolus-related index d is calculated in each segmental artery, these indices are added up in each lobar PA, and thus the related index d in the lobar PA is calculated. In the same manner, these indices are added up in the main PA and thus the related index d in the main PA is calculated, so that, finally, the embolism-related index may be calculated as the Qanadli score in the pulmonary trunk.

FIG. 7 does not limit any embodiment of the present invention, but it shows merely a conceptual diagram showing a process of calculating a pulmonary embolism score and generally known expert knowledge about the structural information of pulmonary vessels that may be used in an embodiment of the present invention. A user interface for assisting pulmonary embolism diagnosis according to an embodiment of the present invention may be provided by combining the conceptual diagram of FIG. 7 with a diagram image (for example, the diagram of FIG. 6) obtained from a medical image of an actual patient.

Referring back to FIG. 6, a pulmonary embolism score is visualized as part of quantitative information, and the quantitative information mapped to each sub-region may include the distribution of pulmonary embolism finding areas within each sub-region. The distribution of pulmonary embolism finding areas may include the sizes of suspected pulmonary embolism areas, and the ratios of the areas affected by the suspected pulmonary embolism areas.

Furthermore, this may also be expanded into a function (a synchronization or invocation function) of visualizing in detail an embolus located in a corresponding vascular sub-region on a diagram through interaction with a vascular diagram.

Another example of the pulmonary embolism score is a Miller score, which uses an objective score and a subjective score. The Miller score approach may define the number of segmental arteries differently from the Qanadli score.

The objective score may be represented by a score for arterial obstruction. It may be calculated only by the presence of a segmental embolism regardless of the degree of obstruction.

The right lung assumes the presence of nine segmental arteries. In this case, three segmental arteries may be located in the upper lobe, two segmental arteries in the middle lobe, and four segmental arteries in the lower lobe.

The left lung assumes the presence of seven segmental arteries. In this case, two segmental arteries may be located in the upper lobe, two segmental arteries in the lingula region, and three segmental arteries in the lower lobe.

Since it is assumed that there are a total of 16 segmental arteries, the highest value of the objective score may be 16 points.

The subjective score may be represented by a score for reduction of the peripheral perfusion.

The subjective score may be calculated by dividing/segmenting each lung into upper, middle, and lower zones, distinguishing/categorizing/classifying the degrees of obstruction in each zone into four levels, and then calculating the degree of obstruction in each zone.

In this case, the criteria for scoring the degree of obstruction are as follows:
0: normal perfusion
1: the perfusion is moderately reduced
2: the perfusion is severely reduced
3: the perfusion is absent

Since a maximum of 3 points may be awarded in each of six zones, the highest value of the subjective score may be 18 points.

Since the Miller score is the sum of the objective score and the subjective score, the highest score may be 16+18=34 points.

Referring to the embodiments of FIGS. 1 to 7, the features of the present invention may be described as follows.

In order to diagnose a pulmonary embolism or determine the treatment effect of an antithrombotic agent or thrombolysis for a patient having a pulmonary embolism, information such as a quantitative embolism volume according to the location of a blood vessel is required.

The conventional technologies focus on detecting the presence/absence of a pulmonary embolism without error in a CT image or the like. Meanwhile, the vascular structure of pulmonary arteries is a structure that spreads out like tree branches. Accordingly, even when visualized as a 3D model, blood vessels appear to be clumped together, so that a problem arises in that it is difficult to determine at a glance the part of a vascular structure in which a pulmonary embolism is located.

In particular, there is a demand for a user interface capable of easily showing the presence/absence of a pulmonary embolism, the size of the pulmonary embolism, and the location of the pulmonary embolism at the segmental artery level. It will be obvious to those skilled in the art that the concept of the present invention may be extended and applied to not only segmental arteries but also sub-segmental arteries and lower intra-lobar arteries in another embodiment of the present invention.

According to an embodiment of the present invention, finding information for the diagnosis of a pulmonary embolism may be categorized/classified and visualized at the level of the segmental arteries of pulmonary vessels.

According to an embodiment of the present invention, there may be provided the user interface that effectively shows the possibility that there is a pulmonary embolism at a specific location of an overall pulmonary vascular structure.

According to an embodiment of the present invention, there may be provided the effective diagnostic assistance user interface that may intuitively convey the location of pulmonary embolism finding information to a medical expert by visualizing the structural information of pulmonary vessels at the level of anatomical knowledge.

According to an embodiment of the present invention, it may be possible to effectively assist the diagnosis of a pulmonary embolism by providing various types of pulmonary embolism finding information including quantitative information together with the structural information of pulmonary vessels to a medical expert.

According to an embodiment of the present invention, there may be provided the user interface for visualizing actual vascular structural information at the level of segmental arteries by using a simplified vascular structure diagram image generated based on the actual vascular structure of a patient.

According to an embodiment of the present invention, diagnosis may be assisted by visualizing the pulmonary embolism finding information of each sub-region using a simplified vascular structure diagram image generated based on the actual vascular structure of a patient.

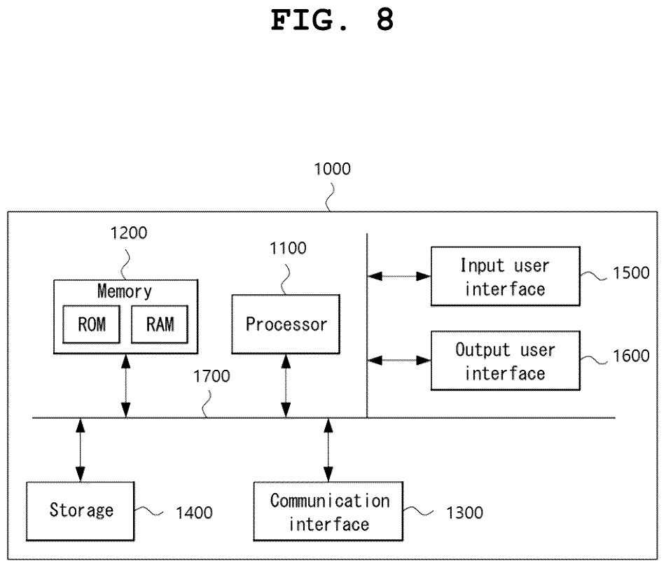

FIG. 8 is a conceptual diagram showing an example of a generalized apparatus for performing medical image processing, analysis and visualization, and diagnosis assistance and visualizing the structural information of pulmonary vessels in medical images, an apparatus for assisting pulmonary embolism diagnosis, or a computing system that is capable of performing at least some of the processes of FIGS. 1 to 7.

At least some of the processes of a method of performing medical image processing, analysis and visualization, and diagnosis assistance and visualizing the structural information of pulmonary vessels in medical images, and a method of assisting pulmonary embolism diagnosis according to an embodiment of the present invention may be performed by the computing system 1000 of FIG. 8.

Referring to FIG. 8, the computing system 1000 according to an embodiment of the present invention may include a processor 1100, memory 1200, a communication interface 1300, storage 1400, an input interface 1500, an output interface 1600, and a bus 1700.

The computing system 1000 according to an embodiment of the present invention includes at least one processor 1100 and memory 1200 configured to store instructions that instruct the at least one processor 1100 to perform at least one step. At least some steps of the method according to an embodiment of the present invention may be performed in such a manner that the at least one processor 1100 loads instructions from the memory 1200 and executes them.

The processor 1100 may refer to a central processing unit (CPU), a graphics processing unit (GPU), or a dedicated processor on which the methods according to embodiments of the present invention are performed.

Each of the memory 1200 and the storage 1400 may be formed of at least one of a volatile storage medium and a non-volatile storage medium. For example, the memory 1200 may be formed of at least one of read-only memory (ROM) and random access memory (RAM).

Furthermore, the computing system 1000 may include the communication interface 1300 that performs communication through a wireless network.

Furthermore, the computing system 1000 may further include the storage 1400, the input interface 1500, and the output interface 1600.

Furthermore, the individual components included in the computing system 1000 may be connected by the bus 1700 and communicate with each other.

Examples of the computing system 1000 of the present invention may include a desktop computer, a laptop computer, a notebook, a smartphone, a tablet personal computer (PC), a mobile phone, a smart watch, smart glasses, an e-book reader, a portable multimedia player (PMP), a portable game console, a car navigation device, a digital camera, a digital multimedia broadcasting (DMB) player, a digital audio recorder, a digital audio player, a digital video recorder, a digital video player, and a personal digital assistant (PDA) that are capable of communication.

An apparatus for visualizing the structural information of pulmonary vessels in medical images according to an embodiment of the present invention may include: memory 1200 configured to store one or more instructions; and a processor 1100 configured to load the one or more instructions from the memory 1200 and execute the one or more instructions. The processor 1100, by executing the one or more instructions, obtains the structural information of pulmonary vessels based on anatomical structure features of the pulmonary vessels from a medical image based on the segmentation results of a pulmonary vascular region of the medical image, obtains pulmonary embolism finding information in the pulmonary vessels of the medical image, visualizes the structural information of the pulmonary vessels in accordance with hierarchical information, and visualizes the pulmonary embolism finding information in association with the individual sub-regions of the structural information of the pulmonary vessels visualized in accordance with the hierarchical information.

The pulmonary embolism finding information visualized in each of the individual sub-regions of the structural information of the pulmonary vessels may include quantitative information about the possibility of a pulmonary embolism diagnosis for each sub-region of the structural information of the pulmonary vessels.

The pulmonary embolism finding information visualized in each of the individual sub-regions of the structural information of the pulmonary vessels may include whether an embolus has been detected in each sub-region of the structural information of the pulmonary vessels and the size of the detected embolus.

The processor 1100, by executing the one or more instructions, may obtain segmentation information for the individual sub-regions including the left main artery, right main artery, lobar arteries and segmental arteries of the structural information of the pulmonary vessels when obtaining the structural information of the pulmonary vessels.

The processor 1100, by executing the one or more instructions, may visualize the individual sub-regions of the structural information of the pulmonary vessels in accordance with hierarchical information ranging up to the left main artery, the right main artery, the lobar arteries, and the segmental arteries when visualizing the structural information of the pulmonary vessels in accordance with hierarchical information.

The processor 1100, by executing the one or more instructions, may obtain the structural information of the pulmonary vessels, based on the anatomical structure features of the pulmonary vessels, based on at least one of 1) whether the pulmonary vessels branch from the main artery trunk toward the segmental arteries and/or 2) the results of comparing the relative sizes of the sub-regions of the pulmonary vessels before the branching and the relative sizes of the sub-regions of the pulmonary vessels after the branching when obtaining the structural information of the pulmonary vessels.

The processor 1100, by executing the one or more instructions, may obtain the structural information of the pulmonary vessels including the distribution of individual segmental arteries by using the segmentation results of the pulmonary vascular region, the segmentation results of the lobes of the lung region of the medical image, and the previously known anatomical structure features of the pulmonary vessels when obtaining the structural information of the pulmonary vessels.

The operations of the method according to the exemplary embodiment of the present disclosure can be implemented as a computer readable program or code in a computer readable recording medium. The computer readable recording medium may include all kinds of recording apparatus for storing data which can be read by a computer system. Furthermore, the computer readable recording medium may store and execute programs or codes which can be distributed in computer systems connected through a network and read through computers in a distributed manner.

The computer readable recording medium may include a hardware apparatus which is specifically configured to store and execute a program command, such as a ROM, RAM or flash memory. The program command may include not only machine language codes created by a compiler, but also high-level language codes which can be executed by a computer using an interpreter.

Although some aspects of the present disclosure have been described in the context of the apparatus, the aspects may indicate the corresponding descriptions according to the method, and the blocks or apparatus may correspond to the steps of the method or the features of the steps. Similarly, the aspects described in the context of the method may be expressed as the features of the corresponding blocks or items or the corresponding apparatus. Some or all of the steps of the method may be executed by (or using) a hardware apparatus such as a microprocessor, a programmable computer or an electronic circuit. In some embodiments, one or more of the most important steps of the method may be executed by such an apparatus.

In some exemplary embodiments, a programmable logic device such as a field-programmable gate array may be used to perform some or all of functions of the methods described herein. In some exemplary embodiments, the field-programmable gate array may be operated with a microprocessor to perform one of the methods described herein. In general, the methods are preferably performed by a certain hardware device.

The description of the disclosure is merely exemplary in nature and, thus, variations that do not depart from the substance of the disclosure are intended to be within the scope of the disclosure. Such variations are not to be regarded as a departure from the spirit and scope of the disclosure. Thus, it will be understood by those of ordinary skill in the art that various changes in form and details may be made without departing from the spirit and scope as defined by the following claims.

The present invention is a technology developed under the sponsorship of the Seoul Business Agency of the Seoul Metropolitan Government (2022 Bio-Medical Technology Commercialization Support Project; BT220014; Development of Pulmonary Embolism and Pulmonary Hypertension Diagnosis Assistance System through Artificial Intelligence-Based CT Image Automatic Analysis).

What is claimed is:

1. A method of visualizing structural information of pulmonary vessels in medical images, the method comprising:
obtaining structural information of pulmonary vessels based on anatomical structure features of the pulmonary vessels from a medical image based on segmentation results of a pulmonary vascular region of the medical image;
obtaining pulmonary embolism finding information in the pulmonary vessels of the medical image;
visualizing the structural information of the pulmonary vessels in accordance with hierarchical information; and
visualizing the pulmonary embolism finding information in association with individual sub-regions of the structural information of the pulmonary vessels visualized in accordance with the hierarchical information.

2. The method of claim 1, wherein the pulmonary embolism finding information visualized in each of the individual sub-regions of the structural information of the pulmonary vessels includes quantitative information about a possibility of a pulmonary embolism diagnosis for each sub-region of the structural information of the pulmonary vessels.

3. The method of claim 1, wherein the pulmonary embolism finding information visualized in each of the individual sub-regions of the structural information of the pulmonary vessels includes whether an embolus has been detected in each sub-region of the structural information of the pulmonary vessels and a size of the detected embolus.

4. The method of claim 1, wherein the obtaining structural information of the pulmonary vessels comprises obtaining segmentation information for each of the individual sub-regions including a left main artery, a right main artery, lobar arteries and segmental arteries of the structural information of the pulmonary vessels.

5. The method of claim 1, wherein the visualizing the structural information of the pulmonary vessels comprises visualizing the individual sub-regions of the structural information of the pulmonary vessels in accordance with hierarchical information ranging up to a left main artery, a right main artery, lobar arteries, and segmental arteries.

6. The method of claim 1, wherein the obtaining the structural information of the pulmonary vessels comprises obtaining the structural information of the pulmonary vessels based on the anatomical structure features of the pulmonary vessels based on at least one of whether the pulmonary vessels have branched from a main artery trunk toward segmental arteries or results of comparing relative sizes of the sub-regions of the pulmonary vessels before the branching and relative sizes of the sub-regions of the pulmonary vessels after the branching.

7. The method of claim 1, wherein the obtaining the structural information of the pulmonary vessels comprises obtaining the structural information of the pulmonary vessels including a distribution of individual segmental arteries by using at least one of the segmentation results of the pulmonary vascular region, segmentation results of lobes of a lung region of the medical image, or previously known anatomical structure features of the pulmonary vessels.

8. The method of claim 1, wherein the pulmonary embolism finding information visualized in each of the individual sub-regions of the structural information of the pulmonary vessels includes a Qanadli score that quantifies a number of emboli associated with each sub-region of the structural information of the pulmonary vessels.

9. The method of claim 1, wherein the pulmonary embolism finding information visualized in each of the individual sub-regions of the structural information of the pulmonary vessels includes a Miller score using an objective score based on a segmental embolism for each sub-region of the structural information of the pulmonary vessels and a subjective score based on reduction of peripheral perfusion.

10. An apparatus for visualizing structural information of pulmonary vessels in medical images, the apparatus comprising:
memory configured to store one or more instructions; and
a processor configured to load the one or more instructions from the memory and execute the one or more instructions;
wherein the processor, by executing the one or more instructions, is configured to:
obtain structural information of pulmonary vessels based on anatomical structure features of the pulmonary vessels from a medical image based on segmentation results of a pulmonary vascular region of the medical image;
obtain pulmonary embolism finding information in the pulmonary vessels of the medical image;
visualize the structural information of the pulmonary vessels in accordance with hierarchical information; and
visualize the pulmonary embolism finding information in association with individual sub-regions of the structural information of the pulmonary vessels visualized in accordance with the hierarchical information.

11. The apparatus of claim 10, wherein the pulmonary embolism finding information visualized in each of the individual sub-regions of the structural information of the pulmonary vessels includes quantitative information about a possibility of a pulmonary embolism diagnosis for each sub-region of the structural information of the pulmonary vessels.

12. The apparatus of claim 10, wherein the pulmonary embolism finding information visualized in each of the individual sub-regions of the structural information of the pulmonary vessels includes whether an embolus has been detected in each sub-region of the structural information of the pulmonary vessels and a size of the detected embolus.

13. The apparatus of claim 10, wherein the processor, by executing the one or more instructions, is further configured to obtain segmentation information for each of the individual sub-regions including a left main artery, a right main artery, lobar arteries and segmental arteries of the structural information of the pulmonary vessels when obtaining the structural information of the pulmonary vessels.

14. The apparatus of claim 10, wherein the processor, by executing the one or more instructions, is further configured to visualize the individual sub-regions of the structural information of the pulmonary vessels in accordance with hierarchical information ranging up to a left main artery, a right main artery, lobar arteries, and segmental arteries when visualizing the structural information of the pulmonary vessels in accordance with the hierarchical information.

15. The apparatus of claim 10, wherein the processor, by executing the one or more instructions, is further configured to obtain the structural information of the pulmonary vessels based on the anatomical structure features of the pulmonary vessels based on at least one of whether the pulmonary vessels have branched from a main artery trunk toward segmental arteries or results of comparing relative sizes of the sub-regions of the pulmonary vessels before the branching and relative sizes of the sub-regions of the pulmonary vessels after the branching when obtaining the structural information of the pulmonary vessels.

16. The apparatus of claim 10, wherein the processor, by executing the one or more instructions, is further configured to obtain the structural information of the pulmonary vessels including a distribution of individual segmental arteries by using the segmentation results of the pulmonary vascular region, segmentation results of lobes of a lung region of the medical image, or previously known anatomical structure features of the pulmonary vessels when obtaining the structural information of the pulmonary vessels.
